(19) United States
(12) Patent Application Publication
(10) Pub. No.: US 2007/0093864 A1
(43) Pub. Date: Apr. 26, 2007

(54) Method for lancing a dermal tissue target site

(76) Inventor: Jerry Thomas Pugh, Santa Rosa, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/256,547

(22) Filed: Oct. 20, 2005

(51) Int. Cl. A61B 17/32

(52) U.S. Cl. 606/181

(57) ABSTRACT

A method for lancing a dermal tissue target site includes contacting a deformable portion of a dermal tissue lancing device cap with the dermal tissue target site such that an outer rim of a deformable distal compression surface establishes initial contact with the dermal tissue target site. In addition, the aforementioned outer rim is furthermost from a longitudinal axis of the cap in comparison to the remainder of the deformable distal compression surface. Subsequently, the cap is urged towards the dermal tissue target site such that the deformable portion deforms and that the remainder of the deformable distal compression surface contacts the dermal tissue target site. The dermal tissue target site is then lanced with the dermal tissue lancing device.

### BACKGROUND OF THE INVENTION

1. Field of the Invention

The present invention relates to medical devices and, in particular, to caps for dermal tissue lancing devices and associated methods.

2. Description of the Related Art

Conventional dermal tissue lancing devices generally have a rigid housing and a lancet that can be armed and launched so as to briefly protrude from one end of the lancing device. For example, conventional lancing devices can include a lancet that is mounted within a rigid housing such that the lancet is movable relative to the rigid housing along a longitudinal axis thereof. Typically, the lancet is spring loaded and launched, upon release of the spring, to penetrate (i.e., “lance”) a target site (e.g., a dermal tissue target site on a user's fingertip). A biological fluid sample (e.g., a whole blood sample) can then be expressed from the penetrated target site for collection and analysis. Conventional lancing devices are described in U.S. Pat. No. 5,730,753 to Morita, U.S. Pat. No. 6,045,567 to Taylor et al. and U.S. Pat. No. 6,071,250 to Douglas et al., each of which is incorporated fully herein by reference.

Dermal tissue lancing devices often include a cap that engages the target site. Such a cap typically has an aperture (i.e., opening), through which the lancet protrudes, and a distal end of the cap will be placed in contact with the target site during use.

When a cap is contacted with a target site, pressure is usually applied to the target site prior to launch of the lancet. This pressure urges the cap against the target site and creates a target site bulge within the opening of the cap. The lancet is then launched to penetrate the target site bulge. A fluid sample, typically blood, is then expressed from the lanced target site for testing. For example, a blood sample expressed from a lanced dermal tissue target site may be tested for the analyte glucose.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings, in which like numerals represent like elements, of which:

FIG. 1 is a simplified perspective view of a cap for a dermal tissue lancing device according to an exemplary embodiment of the present invention;

FIG. 2 is a simplified, perspective, partially cut away view of the cap of FIG. 1 (with dashed lines indicating certain features not visible in the perspective of FIG. 2);

FIG. 3 is a simplified end view of the cap of FIG. 1;

FIG. 4 is a simplified cross-sectional view of the cap of FIG. 1;

FIGS. 5A, 5B and 5C are a sequence of simplified cross-sectional views of the cap of FIG. 1 depicting the cap in the process of being urged against a dermal tissue target site TS and creating a dermal tissue target site bulge B;

FIG. 6 is a simplified cross-sectional depiction of a cap for a dermal tissue lancing device according to another exemplary embodiment of the present invention;

FIG. 7A is a simplified cross-sectional depiction of a cap for use with a dermal tissue lancing device according to a further exemplary embodiment of the present invention as initially contacting a dermal tissue target site (TS);

FIG. 7B is a simplified cross-sectional depiction of the cap of FIG. 7A depicting the cap being urged against the dermal tissue target site (TS);

FIG. 8 is a simplified cross-sectional view of a cap for a dermal tissue lancing device according to yet a further exemplary embodiment of the present invention; and

FIG. 9 is a simplified cross-sectional view of a cap for use with a dermal tissue lancing device according to an additional exemplary embodiment of the present invention;

FIG. 10 is a flow diagram illustrating a sequence of steps in a process according to an exemplary embodiment of the present invention that employs the cap of FIG. 1; and

FIGS. 11A through 11E are simplified cross-sectional views depicting various stages of the process of FIG. 10.

### DETAILED DESCRIPTION OF PREFERRED EMBODIMENTS

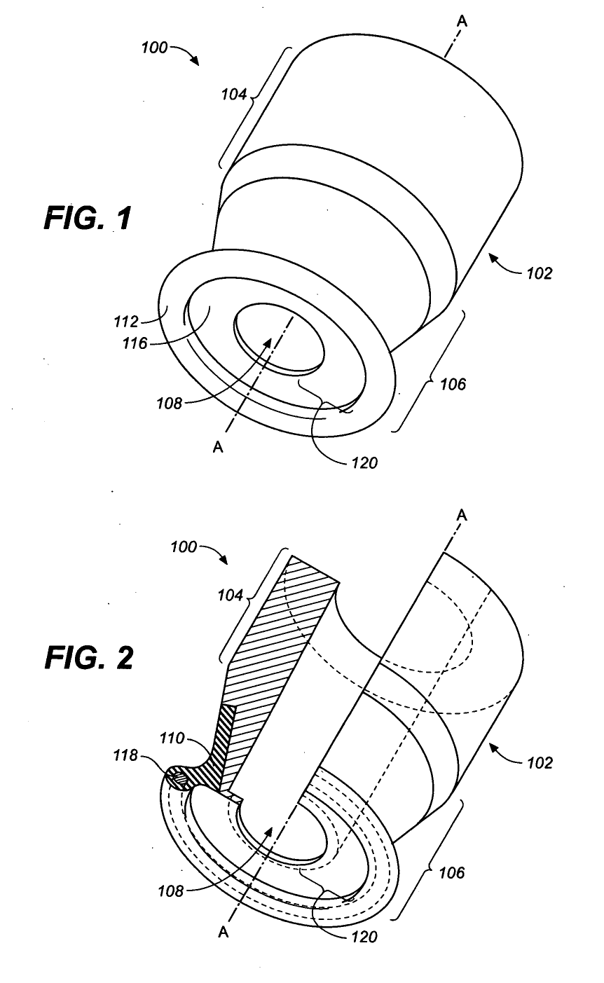

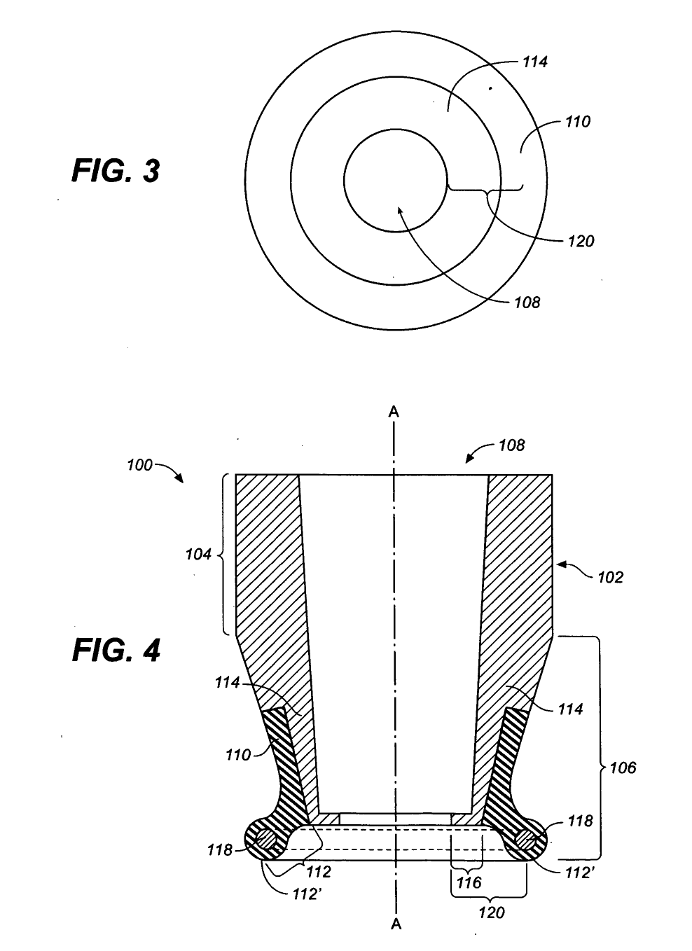

FIG. 1 is a simplified perspective view of a cap 100 for a dermal tissue lancing device (not shown) according to an exemplary embodiment of the present invention. FIG. 2 is a simplified, perspective, partially cut away view of cap 100 (with dashed lines indicating certain features not visible in the perspective of FIG. 2). FIGS. 3 and 4 are a simplified end view of and a simplified cross-sectional view, respectively, of cap 100.

Referring to FIGS. 1, 2, 3 and 4, cap 100 includes a cap body 102 with a proximal end 104, a distal end 106 and opening 108 therethrough. Opening 108 is present along longitudinal axis A-A of cap body 102 (see FIGS. 1, 2 and 4).

Proximal end 104 is configured for engagement with the dermal tissue lancing device. For example, proximal end 104 can be removeably attached to an end of a suitably modified conventional lancing device by slideably mounting, snap-fitting or screw-fitting proximal end 104 to the end of the dermal tissue lancing device. One skilled in the art can readily modify suitable conventional dermal tissue lancing devices for engagement with a proximal end of caps according to embodiments of the present invention. Suitable conventional dermal tissue lancing devices are described in, for example, U.S. Pat. Nos. 5,730,753, 6,045,567 and 6,071,250, each of which is hereby incorporated in full by reference.

However, once apprised of the present invention, one skilled in the art will appreciate that caps according to embodiments of the present invention are not limited to use with the dermal tissue lancing devices described in the aforementioned patents. Rather, caps according to embodiments of the present invention can be used with any suitable dermal tissue lancing device including, for example, those that employ lancets, hollow needles, solid needles, micro-needles, ultrasonic devices, thermal techniques, and any other suitable technique for extraction of a bodily fluid sample from a dermal tissue target site. In addition, the dermal tissue lancing device can, if desired, include an integrated analytical system for the determination of an analyte (e.g., glucose) in an expressed bodily fluid sample.

Distal end 106 is configured for contacting a dermal tissue target site and includes a deformable portion 110 (with a deformable distal compression surface 112, see FIG. 4 in particular), a rigid portion 114 (with a rigid distal compression surface 116, see FIG. 4 in particular). In addition, distal end 106 includes a rigid ring 118 embedded within deformable portion 110. Deformable distal compression surface 112 in combination with rigid distal compression surface 116 is referred to as unified compression surface 120.

As described in detail below, rigid ring 118 serves to maintain a predetermined circumference of cap 100 during use. Rigid ring 118 thereby facilitates a beneficial engagement between cap 100 and a dermal tissue target site as further described below. It should be noted that rigid rings employed in caps according to embodiments of the present invention can be either fully or partially embedded in deformable portion 110 and can take any suitable shape that serves to maintain the aforementioned predetermined circumference of the cap during use.

In the embodiment of FIGS. 1, 2, 3 and 4, rigid portion 114 is integral with proximal end 104. However, one skilled in the art will recognize that rigid portion 114 can be a separate portion.

Opening 108 can have any suitable cross-sectional shape(s) in a direction perpendicular to longitudinal axis A-A including, but not limited to, circular, square, hexagonal, octagonal, and triangular cross-sectional shapes. In addition, the cross-section shape can be such that access to opening 108 by, for example, a test strip is provided. Such test strip access enables beneficial in-situ transfer of a blood sample to the test strip as described in U.S. patent application Ser. No. 10/143,399 (published as US 2003/0143113 A2 on Jul. 31, 2003 and hereby incorporated in full by reference), International Application No. PCT/US01/07169 (published as WO 01/64105 A1 on Sep. 7, 2001) and International Application No. PCT/GB02/03772 (published as WO 03/015627 A2 on Feb. 27, 2003).

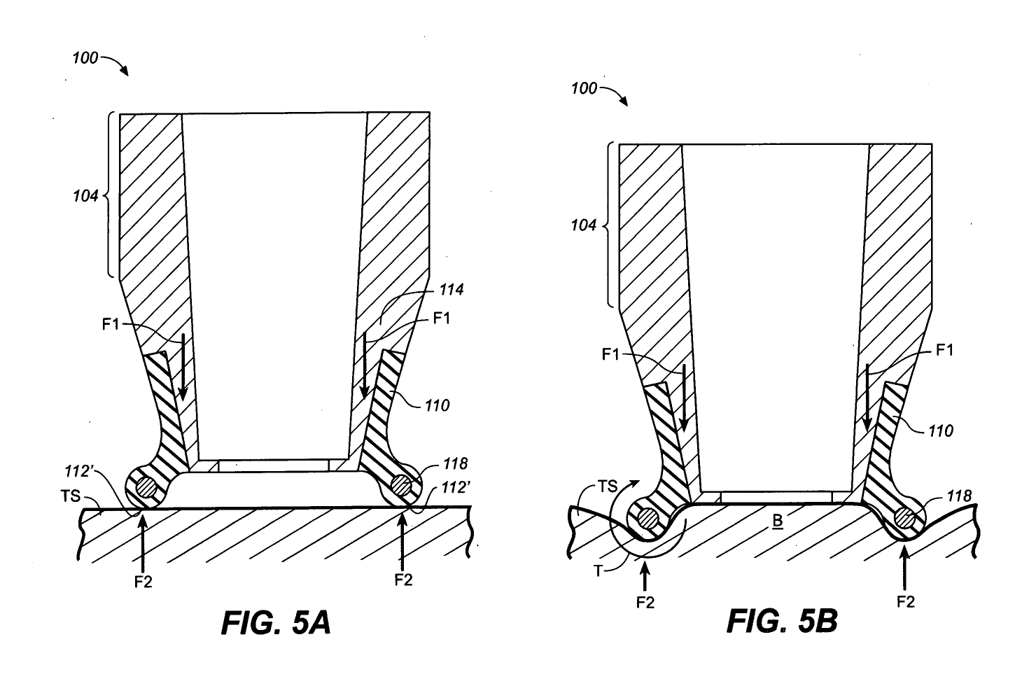

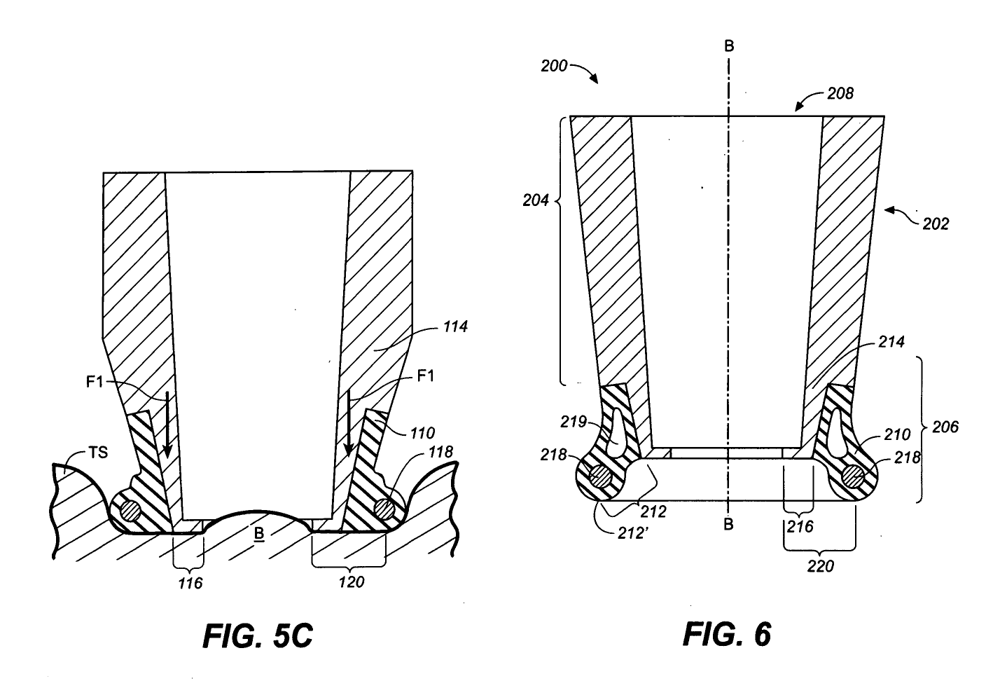

Deformable distal compression surface 112 has an outer rim 112′ (also referred to as an outer edge 112′) that is the furthermost portion of deformable distal compression surface 112 from longitudinal axis. When distal end 106 of cap 100 is contacted with and urged towards a dermal tissue target site, outer rim 112′ establishes initial contact with the dermal tissue target site and, thereafter, deformable portion 110 deforms such that the remainder of deformable distal compression surface 112 and rigid distal compression surface 116 contact the dermal tissue target site. In other words, unified compression surface 120 contacts the dermal tissue target site when cap 100 is urged against the dermal tissue, as will be described further below with respect to FIGS. 5A through 5C.

Deformable portion 110 can be formed of any suitable resiliently deformable material, including, but not limited to, elastomeric materials, polymeric materials, polyurethane materials, latex materials, silicone materials and any combinations thereof. Rigid portion 114 can be formed of any suitable relatively rigid material, including, for example, a rigid plastic material.

When cap 100 is in a relaxed state (i.e., is not in the process of being urged against a dermal tissue target site), unified compression surface 120 (i.e., the combination of deformable distal compression surface 112 and rigid distal compress surface 116) is a concave surface with respect to a plane that is perpendicular to axis A-A and that passes through outer rim 112′. The concave nature of unified compression surface 120, in combination with rigid ring 118 and the deformable characteristics of deformable portion 110, serves to enhance target site bulge creation within opening 108.

FIGS. 5A, 5B and 5C are a sequence of simplified cross-sectional views of cap 100 depicting cap 100 in the process of being urged against a dermal tissue target site TS and creating a dermal tissue target site bulge B.

Referring to FIG. 5A, as cap 100 is urged toward dermal tissue target site TS via application of downward force F1 via rigid portion 114, initially only deformable distal compression surface outer rim 112′ contacts dermal tissue target site TS. As F1 increases, an opposing force F2 is produced by dermal tissue target site TS. Force F2 is distributed around outer rim 112′.

As F1 increases due to further urging of cap 100 toward dermal tissue target site TS, deformable distal compression surface 112 (including outer rim 112′) frictionally engages and holds dermal tissue of dermal tissue target site TS within outer rim 112′, thereby serving to also trap blood within outer rim 112′. As force F1 is increased, dermal tissue target site bulge B forms within opening 108 (see FIG. 5B).

As force F1 continues to increase, deformable portion 110 resiliently deforms, such that rigid portion 114 approach dermal tissue target site TS and rigid distal compression surface 116 contacts the dermal tissue (see FIG. 5C). As F1 is increasing and deformable portion 110 is deforming, rigid ring 118 beneficially serves to maintain the circumference of outer rim 112′ as deformable portion 110 deforms.

As deformable portion 110 is deforming, a force couple F1*F2 produces a torque T (depicted by a curved arrow in FIG. 5B) defined by the following equation:
T=F1*d
where:

d=the moment arm between forces F1 and F2.

Torque T causes deformable portion 118 to deform without changing the position or diameter of outer rim 112′. It should be noted that the size and shape of opening 108 remains unchanged throughout the process depicted in FIGS. 5A, 5B and 5C.

Upon application of a sufficient force F1, a substantially flat unified compression surface 120 is formed (see FIG. 5C), force F2 (not depicted in FIG. 5C) is distributed over said unified compression surface 120 and dermal tissue target site bulge B is fully formed within opening 108.

The deformable nature of deformable portion 110 is beneficial in that deformable portion 110 can move relative to rigid portion 114 (subject to the circumferential restriction provided by rigid ring 118) such that unified compression surface 120, including rigid distal compression surface 116, makes contacts with the dermal tissue target site. Moreover, unified compression surface 120 exerts pressure on dermal tissue target site TS to aid in dermal tissue target site bulge B formation and expression of a blood sample following lancing of dermal tissue target site bulge B.

FIG. 6 is a simplified cross-sectional depiction of a cap 200 for a dermal tissue lancing device (not shown) according to another exemplary embodiment of the present invention. Cap 200 includes a cap body 202 with a proximal end 204, a distal end 206 and opening 208 therethrough. Opening 208 is present along longitudinal axis B-B of cap body 202.

Proximal end 204 is configured for engagement with the dermal tissue lancing device. Distal end 206 is configured for contacting a dermal tissue target site and includes a deformable portion 210 (with a deformable distal compression surface 212), a rigid portion 214 (with a rigid distal compression surface 216). Moreover, distal end 206 also includes a rigid ring 218 and a hollow space 219, both embedded within deformable portion 210. Deformable distal compression surface 212 in combination with rigid distal compression surface 216 serve as a unified compression surface 220.

Rigid ring 218 serves to maintain a predetermined circumference of cap 200 during use. Rigid ring 218 thereby facilitates a beneficial engagement between cap 200 and a dermal tissue target site, as was described with respect to cap 100.

Deformable distal compression surface 212 has an outer rim 212′ (also referred to as an outer edge 212′) that is the furthermost portion of deformable distal compression surface 212 from longitudinal axis B-B. When distal end 206 of cap 200 is contacted with and urged towards a dermal tissue target site, outer rim 212′ establishes initial contact with the dermal tissue target site and, thereafter, deformable portion 210 deforms such that the remainder of deformable distal compression surface 212 and rigid distal compression surface 216 contact the dermal tissue target site. In other words, unified compression surface 220 contacts the dermal tissue target site when cap 200 is sufficiently urged against the dermal tissue target site. Hollow space 219 serves to beneficially increase the deformability of deformable portion 210 during use.

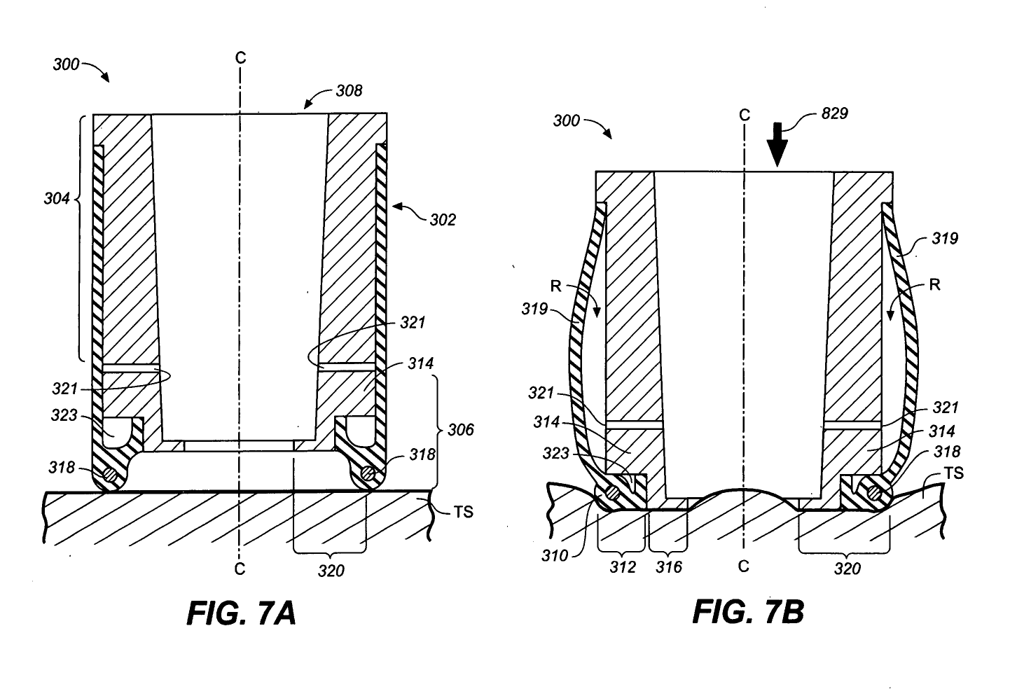

FIG. 7A is a simplified cross-sectional depiction of a cap 300 for use with a dermal tissue lancing device (not shown) according to a further exemplary embodiment of the present invention as initially contacting a dermal tissue target site (TS). FIG. 7B is a simplified cross-sectional depiction of cap 300 depicting cap 300 being urged against the dermal tissue target site (TS).

Cap 300 includes a cap body 302 with a proximal end 304, a distal end 306 and opening 308 therethrough. Opening 308 is present along longitudinal axis C-C of cap body 302.

Proximal end 304 is configured for engagement with the dermal tissue lancing device. Distal end 306 is configured for contacting a dermal tissue target site TS and includes a deformable portion 310 (with a deformable distal compression surface 312), a rigid portion 314 (with a rigid distal compression surface 316), a rigid ring 318 embedded therein, and deformable extension 319 extending along proximal end 304. Deformable distal compression surface 312 in combination with rigid distal compression surface 316 serve as a unified compression surface 320. In addition, proximal end 304 includes vent holes 321 (which are overlain by deformable extension 319) and a gap 323 is present between deformable portion 310 and rigid portion 314.

Rigid ring 318 serves to maintain a predetermined circumference of cap 300 during use. Rigid ring 318 thereby facilitates a beneficial engagement between cap 300 and a dermal tissue target site, as was described with respect to cap 100.

When deformable portion 310 deforms due to the urging of cap 300 against dermal tissue target site TS, gap 323 increased the deformability of deformable portion 310. In addition, as cap 300 is urged against a dermal tissue target site, deformable extension 319 flexes away from the remainder of cap body 302, creating a region R of relatively low pressure (see FIG. 7B) between deformable extension 319 and vent holes 321. Air, therefore, flows through vent holes 321 into region R, and a partial vacuum is formed within opening 308 that beneficially aids in expressing a bodily fluid sample from dermal tissue target site following lancing.

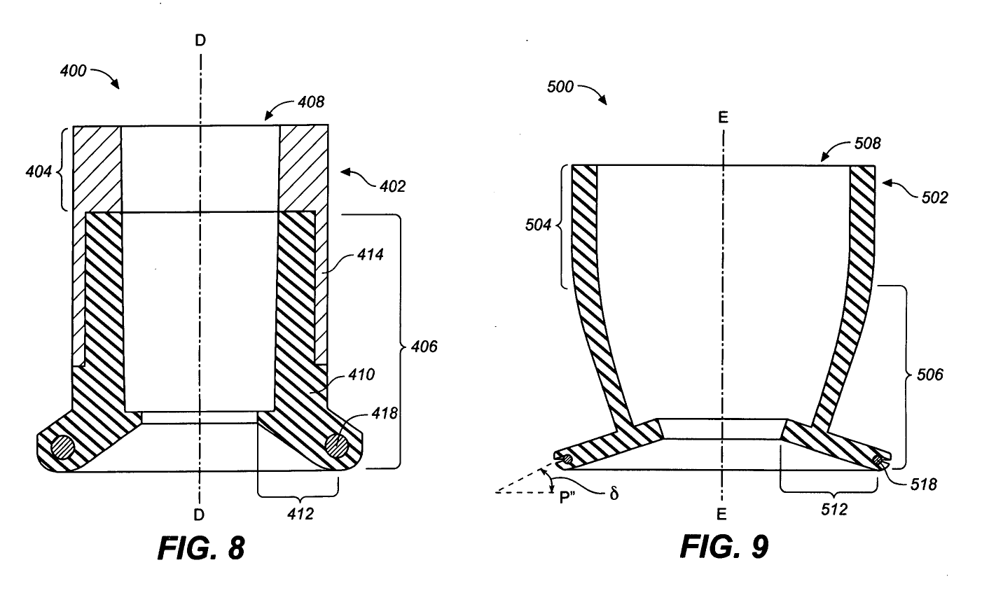

FIG. 8 is a simplified cross-sectional view of a cap 400 for a dermal tissue lancing device (not shown) according to yet a further exemplary embodiment of the present invention. Cap 400 includes a cap body 402 with a proximal end 404, a distal end 406 and opening 408 therethrough. Opening 408 is present along longitudinal axis D-D of cap body 402.

Proximal end 404 is configured for engagement with the dermal tissue lancing device. Distal end 406 is configured for contacting a dermal tissue target site (not shown in FIG. 8) and includes a deformable portion 410 (with a deformable distal compression surface 412), a rigid portion 414, and a rigid ring 418 embedded within deformable portion 410.

Rigid ring 418 serves to maintain a predetermined circumference of cap 400 during use. Rigid ring 418 thereby facilitates a beneficial engagement between cap 400 and a dermal tissue target site, as was described with respect to cap 100.

When deformable portion 410 deforms due to the urging of cap 400 against dermal tissue target site, deformable distal compression surface 412 becomes a flat compression surface operatively engaged with the dermal tissue target site to create a dermal tissue target site bulge within opening 408. It should be noted that a difference between the embodiment depicted in FIG. 8 and previously described embodiments is that cap 400 does not include a rigid distal compression surface.

FIG. 9 is a simplified cross-sectional view of a cap 500 for a dermal tissue lancing device (not shown) according to an additional exemplary embodiment of the present invention. Cap 500 includes a cap body 502 with a proximal end 504, a distal end 506 and opening 508 therethrough. Opening 508 is present along longitudinal axis E-E of cap body 502.

Proximal end 504 is configured for engagement with the dermal tissue lancing device. Distal end 506 is configured for contacting a dermal tissue target site (not shown in FIG. 9) and includes a flat deformable distal compression surface 512 and rigid ring 518. With the exception of rigid ring 518, distal end 506 is formed entirely of a deformable material.

Rigid ring 518 serves to maintain a predetermined circumference of cap 500 during use. Rigid ring 518 thereby facilitates a beneficial engagement between cap 500 and a dermal tissue target site, as was described with respect to cap 100.

Distal end 506 will deform upon urging of cap 500 against dermal tissue target site such that deformable distal compression surface 512 is operatively engaged with the dermal tissue target site to create a dermal tissue target site bulge within opening 508. It should be noted that a difference between the embodiment depicted in FIG. 9 and previously described embodiments is that cap 500, with the exception of rigid ring 518, is formed entirely of deformable material.

Deformable distal compression surface 512 is essentially a flat surface and forms an angle δ with plane P″ perpendicular to longitudinal axis E-E (see FIG. 9). Angle δ ranges from about 25 to 75 degrees when cap 500 is in a relaxed state and becomes essentially zero when cap 500 is sufficiently urged against a dermal tissue target site.

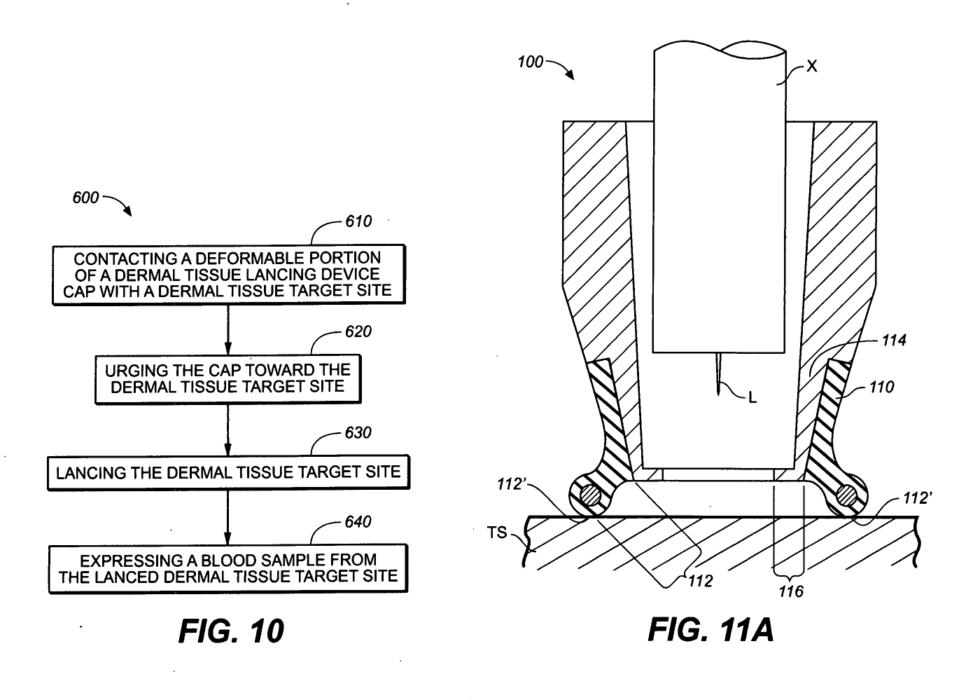

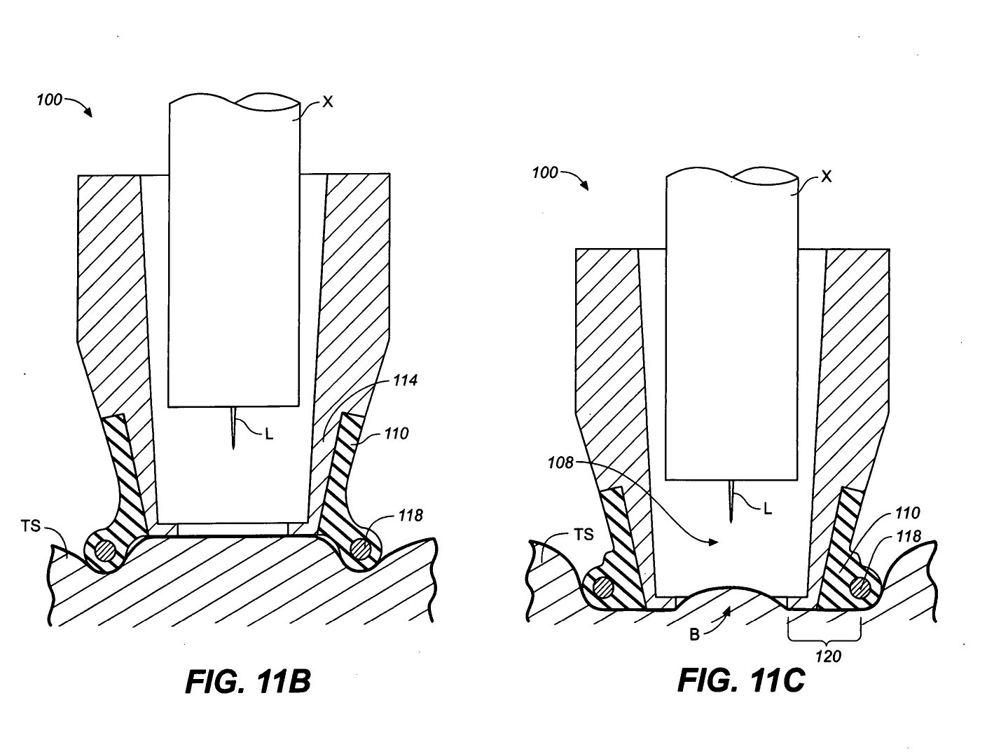

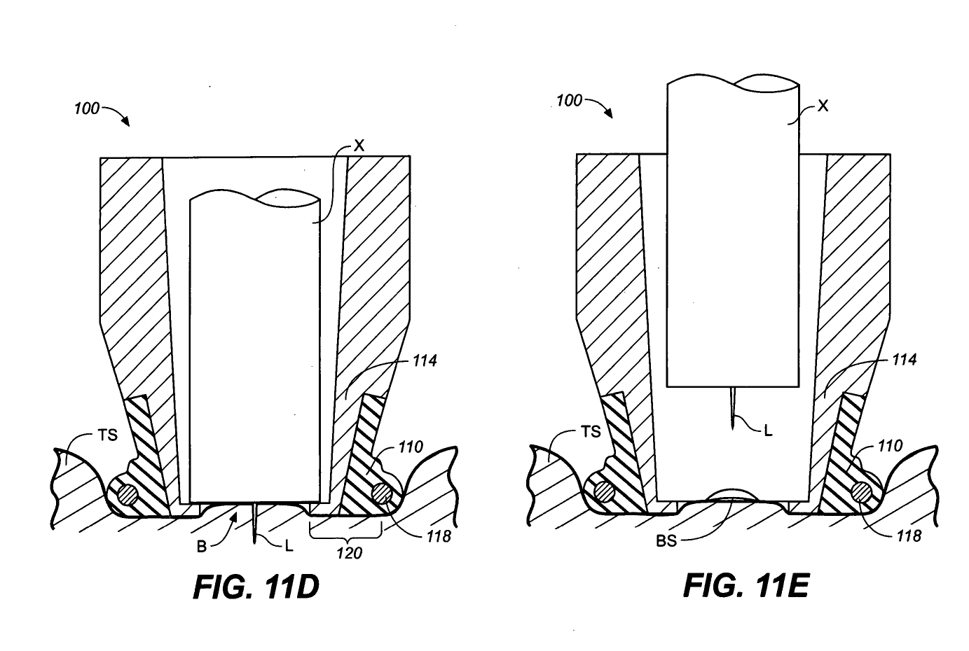

FIG. 10 is a flow chart illustrating a sequence of steps in a process 600 for lancing a dermal tissue target site TS. FIGS. 11A through 11E are simplified cross-sectional views depicting various stages of the process of FIG. 10. For illustrative purposes, cap 100 of FIG. 1 is depicted in FIGS. 11A-11E as being employed in process 600. However, one skilled in the art will recognize that any cap for a dermal tissue lancing device according to the present invention can be employed in methods for lancing a dermal tissue target site according to the present invention. In this regard, it should be noted that any functional behavior of caps for dermal tissue lancing devices according to embodiments of the present invention as described herein can be included in methods for lancing a dermal tissue target site according to the present invention. Moreover, one skilled in the art will recognize that FIGS. 11A through 11E depict only a portion X of a dermal tissue lancing device with portion X including a lancet L.

Process 600 includes contacting deformable portion 110 of dermal tissue lancing device cap 100 with the dermal tissue target site TS such that outer rim 112′ of deformable distal compression surface 112 establishes initial contact with the dermal tissue target site, as set forth in step 610 of FIG. 10 and illustrated in FIG. 11A. As described above with respect to cap 100, the aforementioned outer rim 112′ is furthermost from a longitudinal axis of cap 100 in comparison to the remainder of the deformable distal compression surface 112.

Subsequently at step 620, cap 100 is urged towards dermal tissue target site TS such that deformable portion 110 deforms and that remainder of deformable distal compression surface 112 contacts dermal tissue target site TS. This subsequent urging and deformation is depicted in the sequence of FIGS. 11B and 11C. In addition, FIG. 11C illustrates how the urging has resulted in unified compression surface 120 engaging dermal tissue target site TS and the creation of a dermal tissue target site bulge B within opening 108 of cap 100.

Thereafter, dermal tissue target site bulge B is lanced with lancet L (see FIG. 11D and step 630) and a blood sample (BS) expressed from the lanced dermal tissue target site bulge (see FIG. 11E and step 640).

### EXAMPLE 1

### Percent Success Rate Comparative Study

A comparative study between a cap according to an embodiment of the present invention (i.e., cap 400 of FIG. 8) and a conventional rigid cap was conducted using a 28-gauge lancet available from Becton Dickinson of Franklin Lakes, N.J.

The method of testing comprised pressing the cap body (fitted onto the distal end of a conventional lancing device) against a dermal tissue target site for 3 seconds, lancing with a 28-gauge needle, holding the cap against the dermal tissue target site for 10 seconds, releasing the cap and collecting blood with a calibrated glass capillary pipette.

Cap 500 was tested in conjunction with a finger dermal tissue target site and a forearm dermal tissue target site. The rigid cap was tested only in conjunction with a finger dermal tissue target site.

Success was defined as obtaining at least 0.7 microliters of blood. The percent success rate for cap 500 and a rigid cap are listed in Table 1. Since the dermal tissue target sites were not physically manipulated to enhance blood expression, the test success rates can be considered as representative of worst case results.

TABLE I
Percent Success Rate

| Cap Type | Forearm | Finger |
|---|---|---|
| Cap 500 | 63% | 87% |
| | (n = 101) | (n = 45) |
| Rigid Cap | N/A | 0% |
| | | (n = 48) |

The data of Table 1 indicate that the success rate of cap 500 for finger dermal tissue target sites is significantly improved in comparison to a rigid cap. The data also indicates that cap 500 has a noteworthy success rate for forearm dermal tissue target sites.

### EXAMPLE 2

### Subjective Discomfort Study

During the lancing step in Example 1 for tests conducted on a forearm dermal tissue target site, fifty-four of the subjects rated the amount of discomfort experienced using a subjective scale ranging from 0 to 10. In this subjective scale, a rating of 0 indicated that the subject did not feel any pain during lancing and a rating of 10 indicating that lancing was very painful to the subject. The average subjective score for cap 500 was 1.5. This score indicates that the level of discomfort associated with use of cap 500 is relatively low.

It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for lancing a dermal tissue target site, the method comprising:
contacting a deformable portion of a dermal tissue lancing device cap with the dermal tissue target site such that an outer rim of a deformable distal compression surface of the deformable portion establishes initial contact with the dermal tissue target site, the outer rim being furthermost from a longitudinal axis of the cap in comparison to the remainder of the deformable distal compression surface;
urging the cap towards the dermal tissue target site such that the deformable portion deforms and that the remainder of the deformable distal compression surface contacts the dermal tissue target site; and
lancing the dermal tissue target site with the dermal tissue lancing device.

2. The method of claim 1, wherein the urging step creates a dermal tissue target site bulge within an opening of the cap and the lancing step includes lancing the dermal tissue target site bulge.

3. The method of claim 1, wherein a rigid ring is included in the deformable portion, and
wherein during the urging step, the rigid ring serves to maintain a circumference of the deformable distal compression surface.

4. The method of claim 1 further including the step of expressing a blood sample from the lanced dermal tissue target site.

5. The method of claim 1, wherein the urging step also urges a rigid distal compression surface of the cap against the dermal tissue target site.

6. The method of claim 5, wherein the urging step forms the deformable distal compression surface and the rigid distal compression surface into a unified compression surface in contact with the dermal tissue target site.

7. The method of claim 6, wherein the urging step forms the deformable distal compression surface and the rigid distal compression surface into a flat unified compression surface.

8. The method of claim 1, wherein the urging step creates a region of relatively low pressure between the deformable portion and a rigid portion of the dermal tissue lancing device cap.
